(19) United States
(12) Patent Application Publication
(10) Pub. No.: US 2010/0256542 A1
(43) Pub. Date: Oct. 7, 2010

(54) CAST SHRINK

(76) Inventor: Patricia Nausid, Ketchikan, AK (US)

Correspondence Address: BLACK LOWE & GRAHAM, PLLC, 701 FIFTH AVENUE, SUITE 4800, SEATTLE, WA, 98104, US

(21) Appl. No.: 12/418,486

(22) Filed: Apr. 3, 2009

(51) Int. Cl. A61F 5/00

(52) U.S. Cl. 602/3; 602/7

(57) ABSTRACT

The present invention generally relates to a shrinkable covering that can be used to enclose a cast, splint, bandage, injury, or other affected part of the body in order to protect it from water or other external elements.

### FIELD OF THE INVENTION

This invention relates generally to protective devices used to shield casts, splints, and injured body areas from moisture, dirt, and other environmental elements.

### BACKGROUND OF THE INVENTION

Injured areas of the body sometimes require casts, splints, stitches and bandages to promote healing. These healing devices as well as the injured area itself can be negatively affected when coming in contact with moisture, dirt, and other environmental elements.

Usual remedies to protect casts, splints, and injured body areas are available, but they are bulky, cumbersome, and very expensive. They also have to be continually sterilized to prevent the spread of infection. There is a need for improved options to protect healing devices and injured body areas.

### SUMMARY OF THE INVENTION

This invention in one form comprises a tube, sized and configured to receive and arm, leg, or portion thereof. In one version the tube is fitted with elastic at both ends, with one end smaller in diameter than the other in order to follow the contours of an arm or leg. The elastic serves as an anchor keeping the tube in place. Heat from a common hair dryer or other such source is then applied to the tube causing it to shrink. The tube reacts to heat by becoming form-fitted to the body part within the tube.

This invention in another version comprises a bag, sized and configured to the general shape of a sock. Preferably, elastic surrounds the opening of this shape. This version is designed to receive a foot, with or without a cast. Once fitted, heat from a common hair dryer is applied to the shape. The bag reacts to the heat by shrinking, and becoming form-fitted to the area within the shape.

The various examples of the invention may be formed in varying sizes, lengths and diameters with any possible shape. Due to the inexpensive nature of the materials comprising the make-up of this invention (shrinking film and elastic), this invention is intended to be single use and disposable. This negates the effort necessitated by disinfecting, and eliminates the threat of spreading infection.

### BRIEF DESCRIPTION OF THE DRAWINGS

The preferred and alternative embodiments of the present invention are described in detail below with reference to the following drawings.

FIG. 1 is a representative perspective view of an elasticized tube sized and configured to receive an arm cast, splint or injured area.

FIG. 2 is a representative perspective view of a tube surrounding a wrist and forearm with a cast, splint, or injury.

FIG. 3 is a representative perspective view of a form-fitted elasticized tube surrounding a wrist and forearm after the application of heat.

FIG. 4 is a representative perspective view of a bag open at one end, intended to receive a hand.

FIG. 5 is a representative perspective view of an elasticized bag surrounding a hand with a cast, splint, or injury.

FIG. 6 is a representative perspective view of a form-fitted elasticized bag surrounding a hand with a cast, splint, or injury after the application of heat.

FIG. 7 is a representative perspective view of an elasticized bag open at one end, configured roughly to the shape of an open hand, intended to receive a hand.

FIG. 8 is a representative perspective view of an elasticized bag surrounding a hand, with or without a cast, splint, or bandage.

FIG. 9 is a representative perspective view of a form-fitted elasticized bag surrounding a hand after the application of heat.

FIG. 10 is a representative perspective view of an elasticized tube but sized to receive a leg, ankle, knee or thigh, with or without a cast or splint.

FIG. 11 is a representative perspective view of an elasticized tube surrounding an ankle and calf with a cast, splint, or injury.

FIG. 12 is a representative perspective view of a form-fitted elasticized tube surrounding an ankle and calf after the application of heat.

FIG. 13 is a representative perspective view of an elasticized bag open at one end, configured roughly to the shape of a foot and sized to receive a foot.

FIG. 14 is a representative perspective view of an elasticized bag surrounding a foot with a cast, splint, or injury.

FIG. 15 is a representative perspective view of a form-fitted elasticized bag surrounding a foot after the application of heat.

### DETAILED DESCRIPTION OF THE PREFERRED EMBODIMENT

The present invention generally relates to a shrinkable covering that can be used to enclose a cast, splint, bandage, injury, or other affected part of the body in order to protect it from water or other external elements.

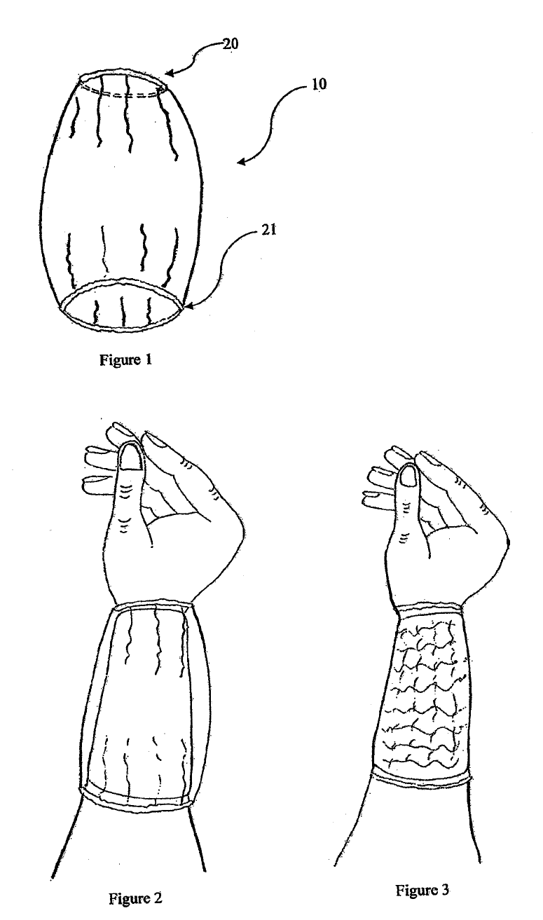

With reference to the figures that follow, common reference numbers are used throughout to refer to common elements. In one example of the invention, the device 10 includes a tube having a first end and a second end, as shown for instance in FIG. 1.

The tube is preferably formed from a plastic film, and in one version the film is made from PVC. Although a variety of thicknesses and sizes will work, PVC film in the range of 50 to 75 gauge (that is, 50 to 75 thousandths of an inch in thickness). Thicker or thinner versions are also suitable, for example, in the range of 25 to 100 gauge. In general, even thicker film construction is possible for the film, so long as it is shrinkable as desired.

PVC film for use with this invention is readily available in a form that is heat-shrinkable, and the shrinkable aspect is a key component of the film that forms the body of the shrinkable covering. While PVC film is used in the preferred form, other versions of plastic or other types of films may also be used to the extent that they are shrinkable. Preferably, film is chosen so that it shrinks to a size within the range of 50 to 90% of its original size, and more preferably toward the upper end of that range.

As further illustrated in FIG. 1, the first end of the preferred tube forming the body of the shrinkable cast covering includes an elastic band 20 along the peripheral edge, and the second end of the body also includes an elastic band 21 along the peripheral edge. The elastic band may be formed from rubber or the like, and can be stitched, sonic-welded, over-molded, glued, or otherwise secured to the edges of the body.

In use, the shrinkable covering is placed over a body part as shown in FIG. 2. In this case, the affected body part is a forearm that is covered by a cast or bandage. Heat is then applied to the shrinkable covering via a hair dryer or other such source in order to shrink the shrinkable covering and conform it relatively closely to the shape and size of the forearm and cast. The elastic bands 20, 21 are preferably sized and shaped such that they fit snugly around the arm in order to generally form a seal against the skin or the cast, as desired. The elastic bands further help to secure the tube body in place during the heat shrinking process in order to facilitate an even, wrinkle-free shrinking.

In other versions of the invention, the elastic bands are not structurally secured to ends of the tube body, but rather are loosely provided along with the tube body and then are placed over the tube body when the tube is placed around the forearm. The elastic bands may then either be left in place after shrinking the film or may be removed, as desired.

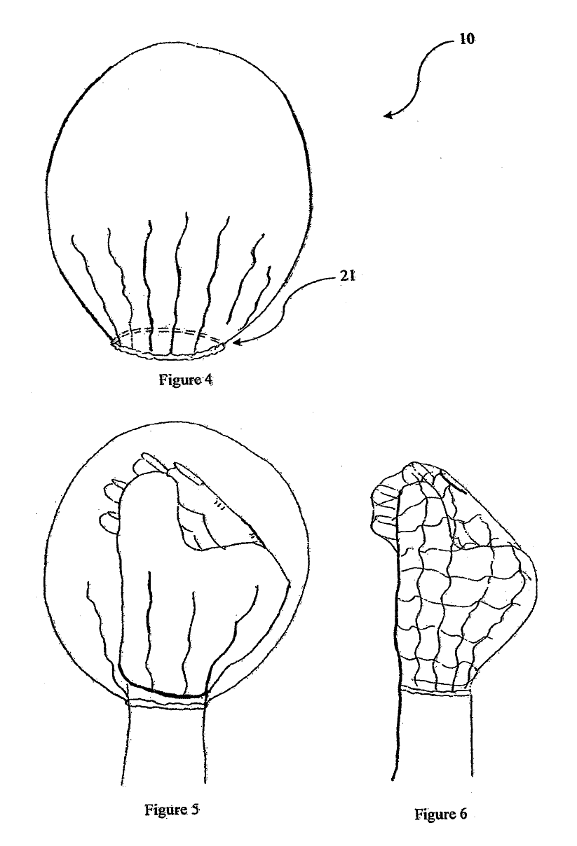

Additional preferred versions of the invention are showed in the remaining Figures. In FIGS. 4-6, a bag-shaped version is shown. Thus, in this example the shrinkable covering body 10 comprises a bag rather than a tube, and therefore there is a single elastic band 21 formed about the open end of the bag.

The bag version is best suited for placement about a hand having an injury or a cast, as is shown in FIG. 5, so that it can be reduced in size with the application of heat as shown in FIG. 6 to snugly form about the hand.

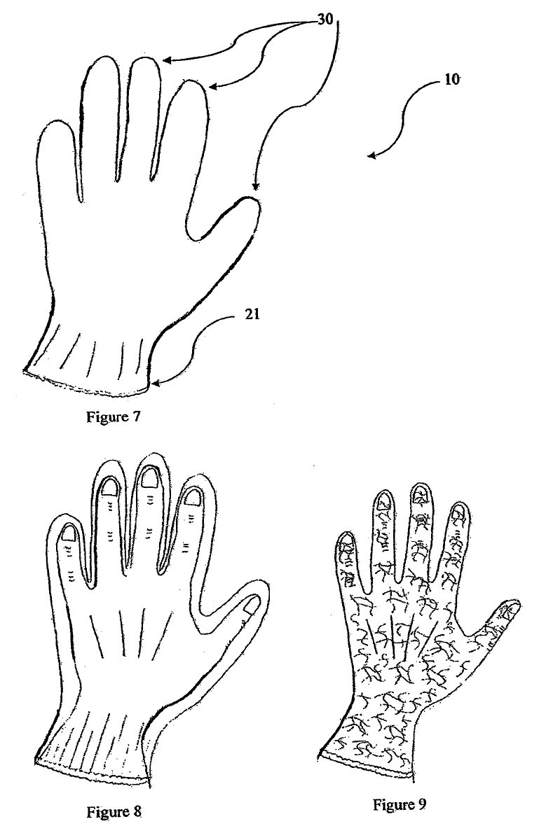

FIGS. 7-9 depict a glove version of the invention in which the shrinkable body 10 includes a plurality of finger-receiving extensions 30 and a single open end having an optional elastic band 21. In the version as shown, the shrinkable body is formed as a standard glove having internal pockets for four fingers and a thumb, with a single opening to receive the hand.

This version is designed to receive a hand, as shown in FIG. 8, and then to be reduced in size by the application of heat in order to closely conform to the shape of a hand, as shown in FIG. 9.

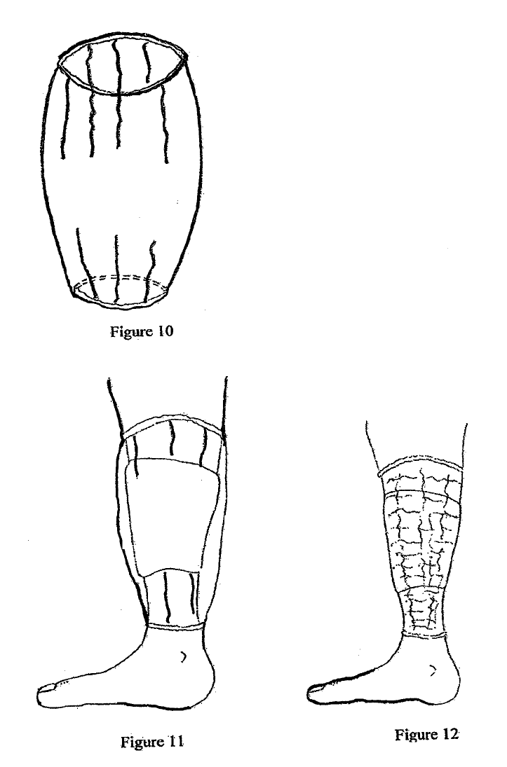

FIGS. 10-12 illustrate a larger version of the tube example of the invention as described above in FIGS. 1-3. While the version of FIGS. 1-3 is sized and shaped to conform to a forearm, the version of FIGS. 10-12 is sized and shaped to conform to a lower leg.

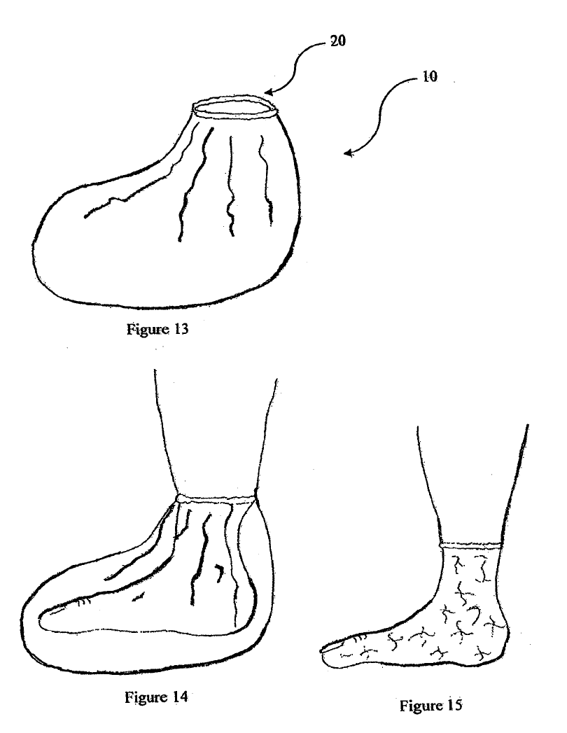

FIGS. 13-15 illustrate a final example in which the shrinkable body 10 is in the form of a bag and includes a single opening having an optional elastic band 20 attached. This version performs and is configured essentially the same as with the version of FIGS. 4-6 except that it is sized and configured to cover a foot and/or ankle rather than a hand.

The above description and drawings show several different versions, and yet additional versions are also possible consistent with this invention, depending on the size and shape of the body part, splint, medical device, etc., that is desired to be covered.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is: claimed are defined as follows:

1. A covering, comprising:
a main body formed from heat-shrinkable material, the main body being sized and configured to cover an affected area, the main body having a first opening to receive the affected area.

2. The covering of claim 1, wherein the heat-shrinkable material comprises PVC.

3. The covering of claim 2, wherein the heat-shrinkable material is between the range of 50 gauge and 75 gauge.

4. The covering of claim 2, further comprising a first elastic band adjacent the first opening.

5. The covering of claim 4, wherein the first elastic band is removably secured about the first opening.

6. The covering of claim 5, wherein the main body further comprises a second opening and a second elastic band removably secured about the second opening.

7. The covering of claim 4, wherein the affected area comprises a cast and the covering is sized and configured to cover the cast.

8. A method for covering an affected region of a human body, comprising:
providing a covering, the covering having a main body formed from heat-shrinkable material, the main body being sized and configured to cover the affected region, the main body having a first opening to receive the affected region;
placing the covering over the affected region such that it substantially covers the affected region; and
shrinking the covering by applying heat to the covering.

9. The covering of claim 8, wherein the heat-shrinkable material comprises PVC.

10. The covering of claim 9, wherein the heat-shrinkable material is between the range of 50 gauge and 75 gauge.

11. The covering of claim 9, further comprising a first elastic band adjacent the first opening.

12. The covering of claim 11, wherein the first elastic band is removably secured about the first opening.

13. The covering of claim 12, wherein the main body further comprises a second opening and a second elastic band removably secured about the second opening.

14. The covering of claim 11, wherein the affected area comprises a cast and the step of placing the covering over the affected region comprises placing the covering such that it covers the cast.
